(19) United States
(12) Patent Application Publication
(10) Pub. No.: US 2026/0335123 A1
(43) Pub. Date: Oct. 29, 2026

(54) ORAL DEVICE AND METHOD OF TREATING SLEEP APNEA

(71) Applicant: The Regents of the University of Michigan, Ann Arbor, MI (US)

(72) Inventors: Jocelyn RA, Ann Arbor, MI (US); Madeline MOUREAU, Ann Arbor, MI (US); Devansh KOHLI, Ann Arbor, MI (US); Mitali Mahesh MAHAJAN, Ann Arbor, MI (US); Idman B. GABAYRE, Ann Arbor, MI (US); Saad SHAMI, Ann Arbor, MI (US); Darwin ARIAS, Ann Arbor, MI (US); Cassie RUIZESPARZA, Ann Arbor, MI (US); Jalen YEN, Ann Arbor, MI (US)

(21) Appl. No.: 19/656,993

(22) Filed: Apr. 23, 2026

Related U.S. Application Data
(60) Provisional application No. 63/794,852, filed on Apr. 25, 2025.

(51) Int. Cl. A61F 5/56

(52) U.S. Cl. CPC A61F 5/566

(57) ABSTRACT

An oral device and method to treat sleep apnea includes a mandibular advancement assembly that is operable to enhance usability and patient comfort. The mandibular advancement assembly has an upper section configured to at least partially overlap an upper row of teeth of a user, a lower section configured to at least partially overlap a lower row of teeth of the user, and a rod coupling the upper section and the lower section. In response to an electronic output indicative of the apneic event from a sensor, the lower section is configured to be advanced from a first position to a second position anterior to the first position and move a lower jaw of the user anteriorly.

### TECHNICAL FIELD

This disclosure relates generally to oral devices and methods of treating sleep apnea.

### BACKGROUND

Sleep apnea is a serious sleep disorder where a person repeatedly stops and starts breathing during sleep due to a partial or complete obstruction of the airway. Obstructive sleep apnea (OSA) is the most common type of sleep apnea affecting millions of individuals. When an individual with OSA falls asleep, their throat muscles relax, causing the airway to narrow or close completely. During sleep, the individual may experience an apneic event where they temporarily stop breathing. Their oxygen levels drop and their brain wakes them up to reopen the airway. This cycle repeats throughout the night. Sleep apnea has many unpleasant symptoms including snoring, gasping and choking during sleep, daytime sleepiness, headaches, difficulty concentrating, mood changes, and dry mouth upon waking. If left untreated, sleep apnea can lead to high blood pressure, diabetes, stroke, cardiovascular diseases, or memory problems, among other complications.

There are some options currently available for treating sleep apnea; however, many of these options are inadequately treating the disorder. A common treatment is a continuous positive airway pressure (CPAP) machine that keeps the airway open by providing air pressure into the airway during sleep. The individual is connected to the CPAP machine by a mask. Air is delivered to the mask and flows into the airway. Although the CPAP machine can alleviate apneic episodes, 50% of CPAP machine treatments fail due to inherent issues including mask discomfort, airflow issues, noise and disruption, complexity of maintenance, and lack of immediate relief. These issues lead to poor adherence and suboptimal outcomes. Some individuals elect to have surgery to remove tissue or correct structural anatomical issues causing their sleep apnea. However, many individuals do not want an invasive procedure. Other treatments include oral appliances, more specifically mandibular adjustment devices (MADs), that continuously position the jaw or tongue in a forward position to keep the airway open. However, 80% of users experience side effects including jaw pain and discomfort, and time spent re-adjusting the jaw to a normal position.

There remains a need for an improved oral device and method to treat sleep apnea that offers enhanced efficiency, greater patient compliance, and improved usability.

### SUMMARY

In accordance with one embodiment, there is provided an oral device to treat sleep apnea comprising a mandibular advancement assembly. The mandibular advancement assembly has an upper section configured to at least partially overlap an upper row of teeth of a user, a lower section configured to at least partially overlap a lower row of teeth of the user, and a rod coupling the upper section and the lower section. A sensor is positioned in the upper section of the mandibular advancement assembly and configured for insertion into an oral cavity of the user posterior of the upper row of teeth, wherein the sensor is configured to generate an electronic output. A controller is electronically coupled to the sensor and configured to receive the electronic output and determine that the electronic output is indicative of an apneic event. In response to the electronic output indicative of the apneic event, the lower section is configured to be advanced from a first position to a second position anterior to the first position and move a lower jaw of the user anteriorly.

In accordance with another embodiment, there is a method of treating sleep apnea. The method includes the step of receiving, at a controller, an electronic output from a senso. The sensor is inserted into an oral cavity of a user posterior of an upper row of teeth of the user. The method includes determining that the electronic output is indicative of an apneic event and actuating a mandibular advancement assembly. The mandibular advancement assembly comprises an upper section configured to at least partially overlap the upper row of teeth, a lower section configured to at least partially overlap a lower row of teeth, and a rod coupling the upper section and the lower section, and wherein, upon actuation, the lower section is advanced from a first position to a second position anterior to the first position to move a jaw of the user anteriorly.

### BRIEF DESCRIPTION OF THE DRAWINGS

Illustrative embodiments will hereinafter be described in conjunction with the following drawings, wherein like numerals denote like elements, and wherein:

FIG. 1 depicts an embodiment of an oral device to treat sleep apnea;

FIG. 2 is a back-side view showing the embodiment of the oral device to treat sleep apnea;

FIG. 3 is a front-side view of the embodiment of the oral device to treat sleep apnea;

FIG. 4 shows an upper liner and a lower liner that can be removably received by the oral device;

FIG. 5 presents a schematic depiction of certain elements inside the housing of the oral device;

FIG. 6 is a representation of the second position of the lower section where the lower jaw of the user is advanced;

FIG. 7 is a representation of the sensor of the oral device generating an electronic output indicative of the apneic event where the throat of the user is closed such that air cannot pass through the airway;

FIG. 8 is a representation of the lower section of the oral device in the second position where the jaw of the user is advanced such that the throat of the user is open and air can pass through the airway; and

FIG. 9 is a flowchart illustrating example method steps for treating sleep apnea.

### DETAILED DESCRIPTION

Referring generally to the drawings, an embodiment of an oral device 10 and method 200 of treating sleep apnea is set forth. The oral device 10 includes a mandibular advancement assembly 12 having an upper section 14, a lower section 16, and a rod 18 coupling the upper section 14 and the lower section 16. The upper section 14 is configured to at least partially overlap an upper row of teeth 20 of a user 22 and the lower section 16 is configured to at least partially overlap a lower row of teeth 24 of the user 22. Movement of the rod 18 connecting the upper and lower sections 14, 16 may effectuate movement of the lower section 16 with respect to the upper section 14. The oral device 10 includes a sensor 26 positioned in the upper section 14 and configured for insertion into an oral cavity 28 of the user 22 posterior of the upper row of teeth 20. Data has shown that this position increases the effectivity of the sensor 26. The oral device 10 includes a controller 30 electronically coupled to the sensor 26 and configured to receive an electronic output from the sensor 26 and determine that the electronic output is indicative of an apneic event. An apneic event may include a cessation of breathing for a period of time. In response, the lower section 16 is configured to be advanced from a first position to a second position anterior to the first position and move a lower jaw 32 of the user 22 anteriorly. Advancing the lower jaw 32 may open the airway 34 of the user 22 such that their breathing can return to normal.

In one embodiment, the method 200 of treating sleep apnea includes receiving, at a controller 30, an electronic output from a sensor 26; determining that the electronic output is indicative of an apneic event; and actuating a mandibular advancement assembly 12. The user’s lower jaw 32 may be advanced to a position where their airway 34 is opened until after the apneic event has terminated. When the electronic output is indicative of termination of the apneic event, the mandibular advancement assembly 12 may be actuated and the lower jaw 32 of the user 22 may be moved posteriorly. In this way, the lower jaw 32 may be only advanced when necessary during an apneic event, eliminating the need for constant mandibular advancement. The automatically-adjusting device 10 and method 200 may significantly reduce discomfort and side effects, including jaw pain and bone restructuring, typically associated with sleep apnea treatments. Further, the device 10 and method 200 may remove the need for morning jaw repositioning devices, leading to a more comfortable and user-friendly experience. Thus, there may be significant potential to improve patient adherence using the device 10 and method 200 disclosed herein, which is notoriously low for obstructive sleep apnea (OSA).

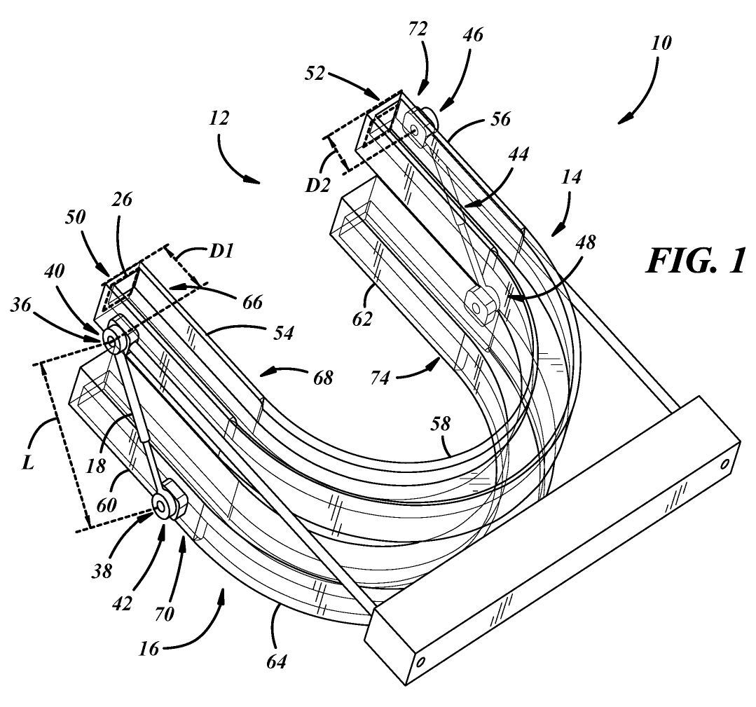

With reference to FIG. 1, an embodiment of an oral device 10 to treat sleep apnea is depicted. The oral device 10 may have various designs and may include more, fewer, or alternative components in various embodiments. In the embodiment shown in FIG. 1, the oral device 10 includes a mandibular advancement assembly 12 having an upper section 14, a lower section 16, and a rod 18 coupling the upper and lower sections 14, 16. The upper section 14 is configured to a least partially overlap an upper row of teeth 20 of a user 22 and the lower section 16 is configured to at least partially overlap a lower row of teeth 24 of the user 22. In this context, at least partially overlap means that the two features share a common area even if they do not completely align. Partial overlap includes an object that covers or lies on top of another object. Here, the upper section 14 configured to at least partially overlap the upper row of teeth 20 means that the upper section 14 may not fully fit the teeth 20 but still cover part of the teeth 20. For example, the upper section 14 may only cover the front upper teeth but not the back upper teeth. In another example, the upper section 14 may not fully snap into place over the upper teeth 20 but may be configured to sit over the teeth 20 when in use. The upper section 14 and lower section 16 may be retainer-like sections that complimentarily mate with the upper and lower rows of teeth 20, 24. In the embodiment of FIG. 1, the upper and lower sections 14, 16 may be universal trays that can be used with any user’s upper and lower rows of teeth 20, 24. The trays may have a hollow interior space configured to at least partially overlap the upper and lower rows of teeth 20, 24. Although the trays may not snuggly fit the teeth, a universal tray may advantageously allow the oral device 10 to be mass produced, leading to a reduction in consumer cost of the device 10. The user 22 may use the universal tray with an inner retainer that complimentarily mates with their teeth.

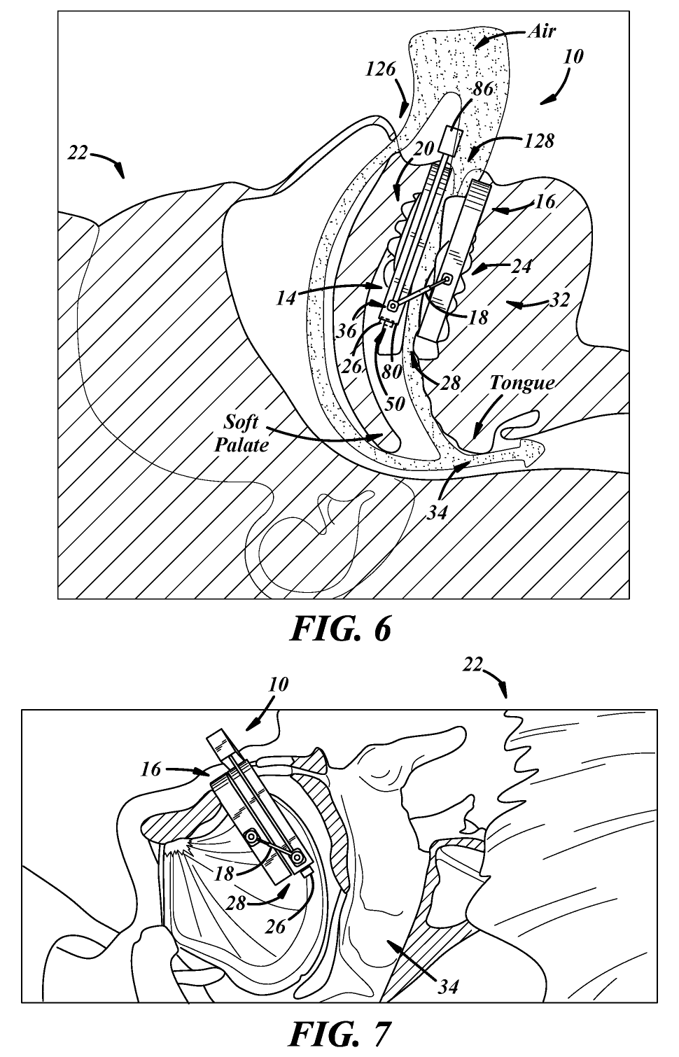

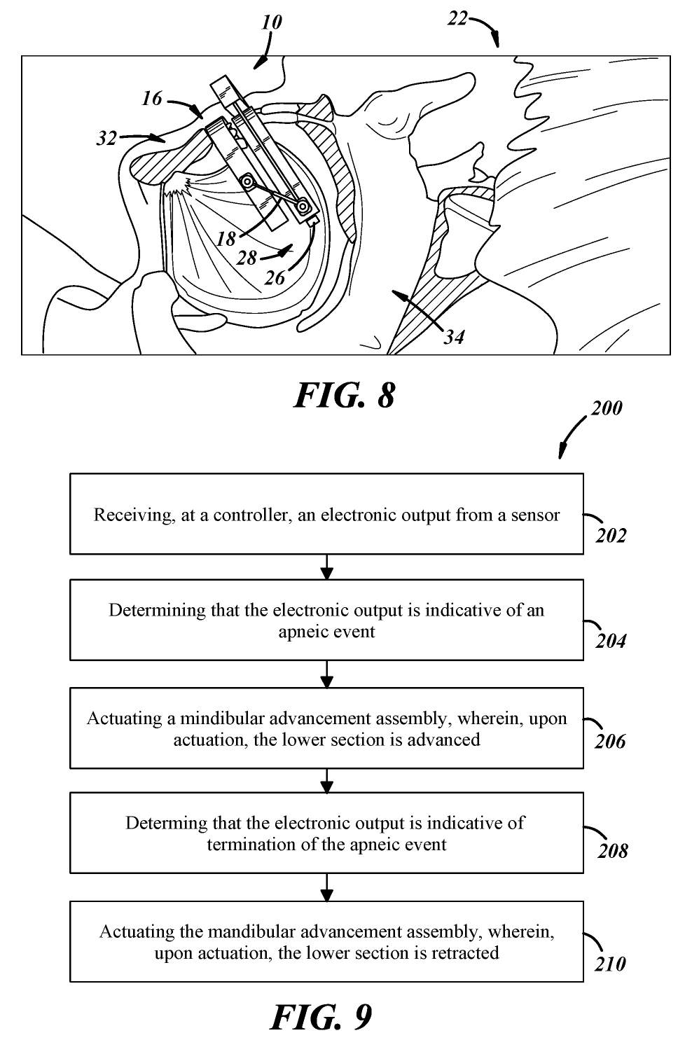

The rod 18 may be coupled to the upper section 14 via a first upper attachment point 36 and coupled to the lower section 16 via a first lower attachment point 38. As used herein, coupled may be used interchangeably with terms such as mounting, attaching, engaging, etc., and may include direct coupling between two subcomponents and/or an indirect coupling between two subcomponents. The rod 18 may extend between the upper and lower sections 14, 16, coupling them. Movement of the rod 18 may effectuate movement of the lower section 16 with respect to the upper section 14. More specifically, the lower section 16 is configured to be advanced from a first position to a second position anterior to the first position and move a lower jaw 32 of the user 22 anteriorly. The first position of the lower section 16 is shown in FIG. 7 and the second position of the lower section 16 is shown in FIGS. 6 and 8.

Movement of the rod 18 may be accomplished using various techniques such as linear movement, rotational movement, vertical movement, articulation, hinging, or any other type of movement. In this context, movement of the rod 18 means changing the location of any part of the rod 18. In one embodiment, the rod 10 may be a telescoping arm comprising multiple arm segments. In those embodiments, the arm is extendable when one arm segment slides out of another arm segment and retractable when one arm segment slides into another arm segment. In this way, the length of the telescoping arm is adjustable. When the arm is extended and the length of the arm is increased, the lower section 16 may be anteriorly advanced from the first position to the second position. When the arm is retracted and the length of the arm is decreased, the lower section 16 may be posteriorly retracted from the second position to the first position, moving the lower jaw 32 of the user 22 posteriorly.

In a preferred embodiment, the rod 18 is a cylindrical structure with a uniform cross-section. The rod 18 may be coupled to the upper section 14 via the first upper attachment point 36. More specifically, in some embodiments, the first upper attachment point 36 may be a first rotation point 36 that the rod 18 is configured to rotate about to anteriorly advance the lower section 16 from the first position to the second position. The first rotation point 36 may be positioned at an upper end 40 of the rod 18. The rod 18 may be coupled to the lower section 16 at the first lower attachment point 38. The first lower attachment point 38 may be positioned at a lower end 42 of the rod 18. In some embodiments, the rod 18 may be configured to rotate about the first lower attachment point 38. Advantageously, the rod 18 may only be configured to rotate about the first rotation point 36 to anteriorly advance the lower section 16. In this preferred embodiment, a length L of the rod 18 is constant as the rod 18 rotates about the first rotation point 36. However, the length L of the rod 18 advantageously impacts the amount that the rod 18 is able to pivot. For example, longer rods may produce a greater pivoting effect, leading to increased mandibular advancement.

In the embodiment of FIG. 1, the rod 18 is a first rod 18 and the mandibular advancement assembly 12 includes a second rod 44 coupling the upper section 14 and the lower section 16. The second rod 44 may be positioned on the opposite side of the mandibular advancement assembly 12 than the first rod 18. For example, the first rod 18 may be configured to be configured to be positioned on the right side of the oral cavity 28 of the user 22 during use of the device 10 and the second rod 44 may be configured to be positioned on the left side of the oral cavity 28. The same information presented in the description of the first rod 18 may be applied to the second rod 44, except where contradictory. In some embodiments, the second rod 44 may be a telescoping arm as with the first rod 18. In this embodiment, the second rod 44 is coupled to the upper section 14 via a second upper attachment point 46. More particularly, in this embodiment the second upper attachment point 46 is a second rotation point 46 that the second rod 44 is configured to rotate about to anteriorly advance the lower section 16 from the first position to the second position. The second rod 44 may be coupled to the lower section 16 at a second lower attachment point 48.

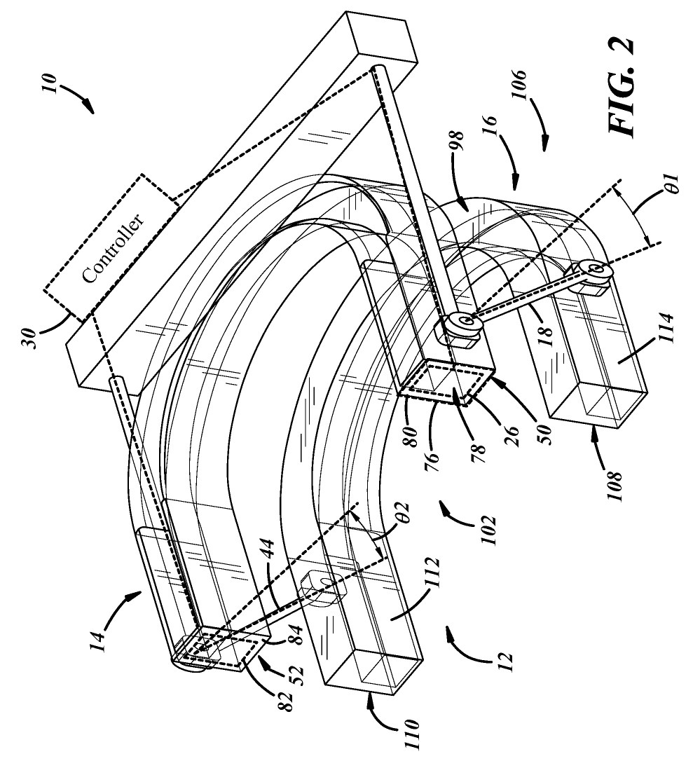

In a preferred embodiment, a distance D1 between the first rotation point 36 and a first posterior wall 50 of the upper section 14 and a distance D2 between the second rotation point 46 and a second posterior wall 52 of the upper section 14 may be the same. Advantageously, the same distance may produce symmetry of the device 10, leading to enhanced patient comfort and increased operability. To advance the lower section 16, the first rod 18 may rotate about the first rotation point 36 a first value of degrees, the second rod 44 may rotate about the second rotation point 46 a second value of degrees, and the first and second values of degrees may be the same. In some embodiments, the first and second rods 18, 44 may be configured to rotate 1-100 degrees, more preferably, 2-90 degrees, more preferably, 2-20 degrees, even more preferably, 2-15 degrees. Advantageously, the angular rotation θ1 of the first rod 18 being the same as the angular rotation θ2 of the second rod 44 (shown in FIG. 2) can help ensure that both sides of the lower jaw 32 are advanced equally, providing comfort to the user 22 and decreasing the chances of the user’s sleep being interrupted by the advancement of the lower jaw 32.

In the embodiment depicted in FIG. 1, the upper section 14 may include a first upper straight segment 54, a second upper straight segment 56, and a third upper curved segment 58 connecting the first upper straight segment 54 and the second upper straight segment 56. The first upper straight segment 54 may be configured to at least partially overlap upper posterior teeth on the right side of the mouth of the user and the second upper straight segment 56 may be configured to at least partially overlap upper posterior teeth on the left side of the user’s mouth. As used herein, a straight segment means that the segment is substantially straight but may include minor curves, bends, or imperfections. Advantageously, the upper straight segments 54, 56 may follow the natural shape of upper posterior teeth that are generally in a substantially straight line. As discussed previously, the upper straight segments may be customized to fit the upper teeth of the user 22 or may be universal so as to at least partially overlap the upper teeth of any user. The third upper curved segment 58 may be configured to at least partially overlap upper anterior teeth of the mouth if the user 22. The third upper curved segment 58 does not need to be a perfectly uniform or continuous curve. For example, the third upper curved segment 58 may have irregularities or changes in curvature to accommodate the anatomy of the user’s upper teeth. The shape and arrangement of the upper segments 54, 56, 58 may advantageously mimic the upper row of teeth 20 of the user 22 such that the device 10 is comfortable to wear in the oral cavity 28. A typical anatomical arrangement of the upper row of teeth 20 may include right posterior teeth that are in a substantially straight line, anterior teeth that are in a substantially curved arrangement, and left posterior teeth that are in a substantially straight line. In a preferred embodiment, the segments 54, 56, 58 of the upper section 14 mimic this anatomical arrangement. In various other embodiments, the shape of the upper section 14 may be adjusted according to user need.

The lower section 16 may include a first lower straight segment 60, a second lower straight segment 62, and a third lower curved segment 64 connecting the first lower straight segment 60 and the second lower straight segment 62. The same information discussed previously regarding the segments 54, 56, 58 of the upper section 14 apply equally here as well except where contradictory. Advantageously, the lower section 16 and the upper section 14 may have the same overall shape because the upper and lower rows of teeth 20, 24 have the same overall arrangement.

The first upper straight segment 54 may have a posterior portion 66 and an anterior portion 68. The posterior portion 66 is posterior to the anterior portion 68 when the device 10 is inside the oral cavity 28 of the user 22. The posterior portion 66 may comprise the posterior half of the first upper straight segment 54 and the anterior portion 68 may comprise the anterior half of the first upper straight segment 54. In other embodiments, the posterior and anterior portions 66, 68 may comprise various percentages of the first upper straight segment 54, for example, the posterior portion 66 may comprise the posterior 25% and the anterior portion 68 may comprise the anterior 75%. Each of the second upper straight segment 56, first lower straight segment 60, and second lower straight segment 62 may have their own posterior portion and anterior portion. The information relating to these portions on the first upper straight segment 54 applies equally here as well except where contradictory.

In some embodiments, the first rod 18 is coupled to the lower section 16 at the first lower attachment point 38 that is anterior of the first rotation point 36 and the second rod 44 is coupled to the lower section 16 at a second lower attachment point 48 that is anterior of the second rotation point 46. This arrangement allows the first and second rods 18, 44 to pivot about the respective first and second rotation points 36, 46 to advance the lower jaw 32 of the user 22. More specifically, in a preferred embodiment, the first rod 18 may be coupled to the upper section 14 via the first rotation point 36 on the posterior portion 66 of the first upper straight segment 54. In those embodiments, the first rod 18 may be coupled to the lower section 16 via the first lower attachment point 38 on an anterior portion 70 of the first lower straight segment 60. This arrangement is advantageous as it allows for a longer rod length L. As discussed previously, the longer rod length L allows for greater pivoting of the rod 18, enhancing the efficiency and operability of the device 10. Similarly, the second rod 44 may be coupled to the upper section 14 via the second rotation point 46 on a posterior portion 72 of the second upper straight segment 56 and coupled to the lower section 16 via the second lower attachment point 48 on an anterior portion 74 of the second lower straight segment 62. In an even more preferred embodiment, the first and second rods 18, 44 may be coupled to the upper section 14 at the most posterior points of the posterior portions 66, 72 of the upper straight segments 54, 56 and coupled to the lower section 16 at the most anterior points of the anterior sections 70, 74 of the lower straight segments 60, 62 to achieve the longest rod possible in this arrangement. This embodiment would further enhance efficiency and operable of the device 10 by allowing the most amount of pivot.

In some embodiments where the rod 18 is configured to rotate, the rod 18 is configured to anteriorly advance the lower section 16 from the first position to the second position by rotating counterclockwise about the first rotation point 36. In embodiments where the first rod 18 and the second rod 44 are configured to rotate, the lower section 16 may be anteriorly advanced by the first rod 18 rotating counterclockwise about the first rotation point 36 and the second rod 44 rotating clockwise about the second rotation point 46. The direction of rotation is determined from the vantage point of viewing the exterior of the device 10 from the side in which the rod 18, 44 is positioned. Advantageously, the direction of rotation of the first rod 18 and the second rod 44 are opposite, ensuring that the lower jaw 32 is efficiently advanced.

The device 10 may be configured to posteriorly retract the lower section 16 from the second position to the first position to move the lower jaw 32 posteriorly. To accomplish this movement of the lower section 16, the rod 18 may rotate clockwise about the first rotation point 36. In embodiments where the first rod 18 and the second rod 44 are configured to rotate, the lower section 16 may be posteriorly retracted by the first rod 18 rotating clockwise about the first rotation point 36 and the second rod 44 rotating counterclockwise about the second rotation point 46. As explained previously, the direction of rotation of the first rod 18 and the second rod 44 is advantageously opposite when retracting the lower section 16 to ensure that the lower jaw 32 is efficiently retracted. Further, the first rod 18 may rotate in opposite directions to advance and retract the lower section 16, for example, counterclockwise to advance the lower section 16 and clockwise to retract the lower section 16. The second rod 44 may also rotate in opposite directions to advance and retract the lower section 16, and the opposite direction of the first rod 18 during each of the advancement and retraction. For example, the second rod 44 may rotate clockwise to advance the lower section 16 while the first rod 18 rotates counterclockwise and the second rod 44 may rotate counterclockwise to advance the lower section 16 while the first rod 18 rotates clockwise. In preferred embodiments, the rate of rotation of the first rod 18 and the second rod 44 may be the same as one another. Further, in those embodiments, the rate of rotation of the rods 18, 44 when advancing the lower section 16 may be the same as the rate of rotation of the rods 18, 44 when retracting the lower section 16.

With reference to FIG. 2, a back-side view of the embodiment of the oral device 10 to treat sleep apnea is shown. A sensor 26 is positioned in the upper section 14 of the mandibular advancement assembly 12. The sensor 26 is configured to generate an electronic output. In some embodiments, the sensor 26 is a transducer. In particular, the sensor 26 may be configured to convert mechanical energy via vibrations created by airflow in the oral cavity 28 to the electronic output. In another example, the sensor 26 may respond to pressure changes as a result of the user 22 breathing and convert that mechanical energy into electrical energy to generate the electronic output. Advantageously, the sensor 26 may be a passive sensor meaning that it responds to an external signal without needing an internal power source to generate the output. In a preferred embodiment, the sensor 26 generates the electronic output in response to vibrational airflow-induced mechanical stress. The sensor 26 may be a piezoelectric sensor that converts mechanical stress from vibrations created by the airflow when the user 22 sleeps to an electric signal. When the material of the sensor 26 is vibrated, it may cause internal displacement of electric charges resulting in a voltage across the sensor 26. The voltage may be sent to a controller 30 to be used in determining whether an apneic event has occurred. Advantageously, no external power is needed to generate the electronic output which eliminates the need for electronics to be placed within the oral cavity 28 of the user 22. Housing the electronics externally of the oral cavity 28 reduces degradation of the electronics and the spread of infection, enhances safety and biocompatibility of the device 10, and reduces regulatory hurdles to implementation, specifically, reduces the requirements for FDA approval.

The sensor 26 may advantageously be a piezoelectric sensor. In a preferred embodiment, the sensor 26 is a polyvinylidene fluoride (PVDF) sensor attached to the upper section 14. A PVDF sensor may be advantageous due to its flexibility and ability to be incorporated into a thin film 76. In the oral device 10, it may be necessary to include the sensor 26 in a specific position only possible where the sensor 26 is within the thin film 76. Further, a PVDF sensor may be highly sensitive to vibrational changes in the airflow of the user 22. The sensor 26 may include a coating 78, particularly, a biocompatible coating, such as a silicone coating or any other coating that is biocompatible. The coating 78 may advantageously protect the sensor 26 from damage, enhance performance of the sensor 26, and improve compatibility with the environment in which the sensor 26 is to be used, for example, the oral cavity 28 of the user 22. Additionally, the biocompatible coating may advantageously ensure compatibility with bodily tissues, maintain sensor 26 functionality, and preserve the integrity of the sensor 26 in the oral cavity 28.

The sensor 26 can be configured for insertion into the oral cavity 28 of the user 22 posterior of the upper row of teeth 20. This position increases the effectivity and operability of the device 10. In some embodiments, because the sensor 26 responds to vibrations from the airflow of the user 22, position of the sensor 26 may affect how effectively it responds. For example, a sensor 26 positioned in the oral cavity 28 near the lips may experience interference due to its proximity to multiple teeth and other oral features leading to a less effective response. Positioning the sensor 26 posterior to the upper row of teeth 20 may increase effectivity of the sensor 26 by reducing interference from other oral features and placing the sensor 26 closer to the airway 34 of the user 22 which, as previously, increases effectivity because the sensor 26 can respond more quickly to a constricted or completely closed airway 34.

In a preferred embodiment, the sensor 26 is positioned adjacent to the posterior wall 50 of the upper section 14. This position may be posterior of the upper row of teeth 20 which may be advantageous for a number of reasons, including, but not limited to, enhanced integrity of sensor readings. More preferably, the sensor 26 may be mounted to an exterior surface 80 of the posterior wall 50 of the upper section 14. In these embodiments, the sensor 26 is advantageously the thin film 76 that can be mounted on the posterior wall 50. In this position, the sensor 26 is configured for exposure to the oral cavity 28 of the user 22. It is preferred that the sensor 26 be exposed to the oral cavity 28 rather than incorporated within the interior of the device 10 so that the sensor 26 can more effectively respond to vibrations from the airflow of the user 22 without interference. The sensor 26 being mounted on the exterior surface of the device 10 may lead to greater exposure to these vibrations. Further, the sensor 26 being mounted on the posterior wall 50 of the upper section 14 may position the sensor 26 closer to the airway 34 of the user 22, increasing the effectivity of the sensor 26.

In some embodiments, the sensor 26 is a first sensor 26 and the oral device 10 includes a second sensor 82 positioned in the upper section 14 of the mandibular advancement assembly 12 and configured for insertion into the oral cavity 28 of the user 22 posterior of the upper row of teeth 20. The information regarding the first sensor 26 as discussed previously applies equally to the second sensor 82 except where contradictory. As with the first sensor 26, the second sensor 82 may be configured to generate an electronic output. The first sensor 26 may be mounted to the exterior surface 80 of the first posterior wall 50 of the upper section 14 and the second sensor 82 may be mounted to an exterior surface 84 of the second posterior wall 52 of the upper section 14. In this arrangement, the first and second sensors 26, 82 are configured for exposure to the oral cavity 28 of the user 22. As discussed previously, exposing the sensors 26, 82 directly to the oral cavity 28 and positioning them posterior to the upper row of teeth 20 increases the effectivity of the sensors 26, 82 by reducing interference and efficiently responding to vibrations near the airway 34 of the user 22.

As shown in FIG. 2, a controller 30 is electronically coupled to the sensor 26 and configured to receive the electronic output and determine that the electronic output is indicative of an apneic event. In some embodiments, the controller 30 may be electronically coupled to the second sensor 82 and configured to receive the electronic output from the second sensor 82 and determine that the electronic output from the second sensor 82 is indicative of the apneic event. Any type of operable controller may be used. Preferably, the controller 30 is configured for use with a piezoelectric sensor. Further, the controller 30 may advantageously be configured to detect electronic output from a passive sensor responding to vibration, analyze the electronic output received, and initiate an action such as advancing the lower section 16 from the first position to the second position.

In this context, an apneic event includes a partial or complete cessation of breathing of the user 22 during sleep that lasts for a period of time. In some embodiments, the period of time may be greater than 3 seconds, more preferably, greater than 5 seconds, even more preferably, greater than 7 seconds. In some cases, the cessation of breathing may lead to a drop in oxygen levels of the user 22. Users with OSA may experience multiple apneic events per night and some may experience multiple apneic events per hour. Typically, the brain triggers the user 22 to fully or partially regain consciousness so that user 22 can resume breathing. The constant arousal from sleep during an apneic event disrupts a user’s nighttime sleep, sometimes multiple times in a single night. The controller 30 advantageously receives the electronic output from the sensor 26 and can use that information to determine whether an apneic event has occurred or is occurring to treat the sleep apnea during or shortly after an apneic event. The determination of the controller 30 that the electronic output from the sensor 26 is indicative of an apneic event allows the device 10 to advantageously treat the sleep apnea before the brain attempts to wake the user 22 up, leading to more continuous sleep during the night.

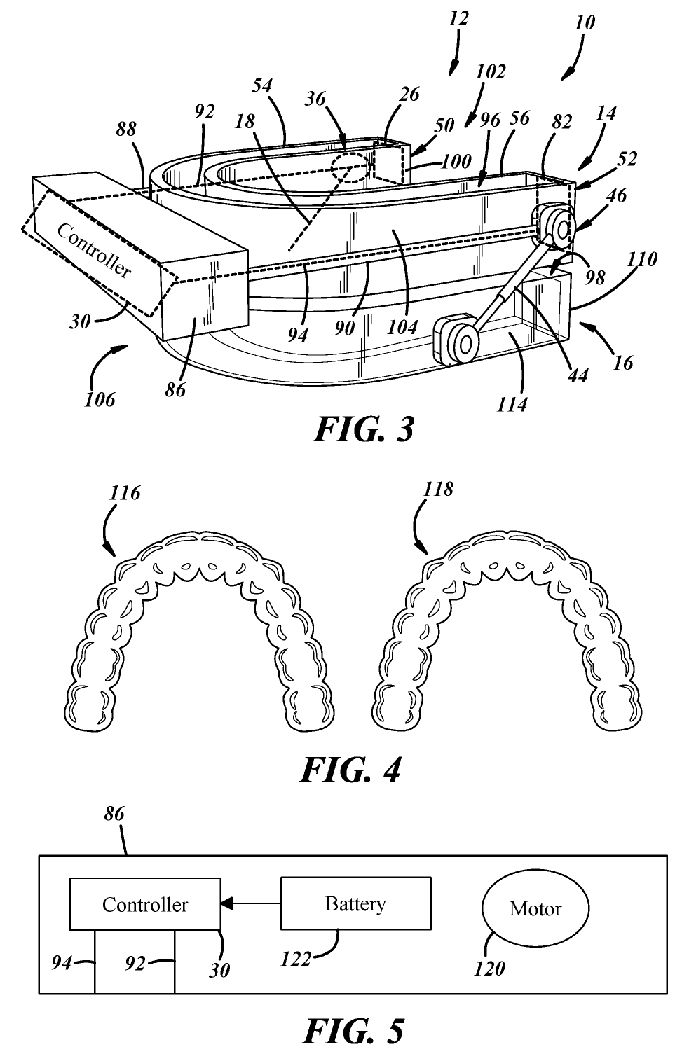

With reference to FIG. 3, a front-side view of the embodiment of the oral device 10 to treat sleep apnea is shown. The device 10 may include a housing 86 operably connected to the mandibular advancement assembly 12. In some embodiments, the housing 86 may enclose and support the electronics of the device 10 in an internal compartment of the housing 86. The housing 86 itself may protect the electronic components from environmental damage, mechanical stress, or other external factors. Further, the internal compartment of the housing 86 may hold the electronic components in place and prevent them from moving or being dislodged during use of the device 10. The housing may 86 be substantially rectangular in shape. In some embodiments, the housing 86 may fully enclose the electronic components. Advantageously, the housing 86 may be configured to be positioned external to the oral cavity 28 of the user 22. In preferred embodiments, the housing 86 may include the electronics of the device 10. As discussed previously, it may be advantageous to position the electronics of the device 10 external to the oral cavity 28 for safety and regulatory reasons.

The housing 86 may also be a handle configured for the user 22 to grasp. In some embodiments, the handle may be designed to fit the hand of the user 22. Particularly, the handle may include a textured grip pattern such as bumps, ridges, or the like, designed to fit the natural shape of the hand. Further, the handle may include indentations configured to receive fingers of the user 22. The user 22 may grasp the handle and use it for insertion and removal of the device 10 into and from the oral cavity 28. Advantageously, the handle is strong enough so that the user 22 may grasp the handle, pick up the device 10, and insert the mandibular advancement assembly 12 into their mouth without damaging the device 10. In this way, the housing 86 may have a duality of functions as both an enclosure for housing electronic components and a handle configured to be grasped by the user 22 for insertion and removal of the device 10 into and out of the oral cavity 28.

The housing 86 may be connected to the upper section 14 via a tube 88. In some embodiments, the housing 86 may be connected to the upper section 14 via two tubes 88, 90, one on each side of the upper section 14. The housing 86 may be connected to the upper section 14 via the first rotation point 36 on the first upper straight segment 54 of the upper section 14. The housing 86 may also be connected to the upper section 14 via the second rotation point 46 on the second upper straight segment 56 of the upper section 14. The tube 88 may be a cylindrical structural with a uniform cross-section. In a preferred embodiment, the tube 88 is a hollow tube with an interior region. The tube 88 may include a wire 92 connecting the sensor 26 to the controller 30. The wire 90 may be disposed within the interior region of the hollow tube 88. In an embodiment where the device 10 includes two tubes 88, 90 connecting the housing 86 to the upper section 14, the tubes 88, 90 may be the same and may each include a wire 92, 94 connecting both the first sensor 26 and the second sensor 82 to the controller 30. Advantageously, the sensor 26, 82 is in physical connection with the controller 30, for example, through the wire 92, 94. The physical connection allows the sensor 26, 82 to be a passive sensor that does not need an internal power source to operate. Other arrangements are certainly possible, including the use of wireless subcomponents, for example.

In a preferred embodiment, the first sensor 26 and the second sensor 82 are positioned in the upper section 14 of the mandibular advancement assembly 12 and are connected to the controller 30 via the wires 92, 94. In that embodiment, the controller 30 is positioned within the housing 86 configured to be positioned external to the oral cavity 28. Still in that embodiment, the housing 86 is connected to the mandibular advancement assembly 12 via tubes 88, 90 which the wires 92, 94 are disposed within at the first rotation point 36 and the second rotation point 46. This embodiment is advantageous because it positions the sensors 26, 82 in an effective position as discussed above while ensuring that the electronics are positioned outside of the oral cavity 28. Having both sensors 26, 82 in the same section of the mandibular advancement assembly 12 is preferred because it allows the same tubes 88, 90 and housing 86 to be used to connect both sensors 26, 82 to the controller 30. Further, positioning the sensors 16, 82 and tubes 88, 90 in the upper section 14 is preferred because the rotation points 36, 46 are located at the upper section 14, allowing the controller 30 to trigger an action to advance the rods 18, 44 by rotating them about the rotation points 36, 46. This arrangement allows all the components to function efficiently and enhances the effectivity of the device 10, but it is feasible to have more or less sensors that what is particularly illustrated.

The upper section 14 may include an interior 96. In some embodiments, the lower section 16 may include an interior 98. The interiors 96, 98 of the sections 14, 16 may comprise a space between outer boundary walls of the section 14, 16. For example, the upper section 14 may include the first posterior wall 50, the second posterior wall 52, an inner sidewall 100 connecting the first and second posterior walls 50, 52 on an inner side 102, and an outer sidewall 104 connecting the first and second posterior walls 50, 52 on an outer side 106. In the embodiment of FIG. 3, the posterior walls 50, 52 and sidewalls 100, 104 create a substantially U-shaped interior 96 of the upper section 14. As shown in FIGS. 2 and 3, the lower section 16 may have a similar interior 98 created by its own first posterior wall 108, second posterior wall 110, inner sidewall 112 connecting the first and second posterior walls 108, 110 on the inner side 102, and outer sidewall 114 connecting the first and second posterior walls 108, 110 on the outer side 106. The interiors 96, 98 may be configured to at least partially overlap the upper and lower row of teeth 20, 24 of the user 22. In some embodiments, the upper and lower rows of teeth 20, 24 of the user 22 may directly contact the interiors 96, 98 during use of the device 10. In other embodiments, the upper and lower rows of teeth 20, 24 may not be in direct contact with the interiors 96, 98 such that another feature separates the teeth 20, 24 from the interiors 96, 98.

With reference to FIG. 4, an upper liner 116 and a lower liner 118 that can be removably received by the oral device 10 are shown. In some embodiments, the device 10 includes the upper liner 116 that can be removably received by the interior 96 of the upper section 14 and the lower liner 118 that can be removably received by the interior 98 of the lower section 16. The upper liner 116 may be configured to mate with the upper row of teeth 20 and the lower liner 118 may be configured to mate with the lower row of teeth 24. The liners 116, 118 may be retainer-like liners that snap into place around the teeth 20, 24 of the user 22. In some embodiments, the user 22 may place the liners 116, 118 onto their teeth 20, 24 then place the mandibular advancement assembly 12 over the liners 116, 118 such that the liners 116, 118 are positioned within the interior 96, 98 of the upper and lower sections 14, 16 during use of the device 10. The upper and lower sections 14, 16 may include an attachment mechanism to attach the liners 116, 118 to the upper and lower sections 14, 16 and hold them in place during use of the device 10. For example, the upper section 14 may include an interlocking or snap-fit connection, a clasp, magnetic coupling, or any other attachment mechanism to receive the upper liner 116. The same attachment mechanism may be used to attach the lower liner 118 to the lower section 16.

In a preferred embodiment, the upper and lower sections 14, 16 are universal sections such that they may be used in connection with any user. The upper and lower liners 116, 118 may be customizable, meaning they are custom-fitted to the user’s teeth 20, 24 or other anatomical features. These liners 116, 118 may be created by taking a dental impression of the user 22 using, for example, a mold or digital scan, and crafting the liners 116, 118 from that impression. The dental impression may ensure a tight fit of the customizable liners 116, 118 such that the liners 116. 118 remain in place during use of the device 10. The liners 116, 118 may be replaced periodically by the user 22 due to ordinary wear-and-tear or upon a change in the dental anatomy of the user 22, such as, for example, if the user 22 loses a tooth or experiences other tooth or jaw movement. In this preferred embodiment, the universal upper and lower section 14, 16 can be kept by the user 22 indefinitely while periodically replacing the liners 116, 118, reducing cost for the user 22 and enhancing the comfortability of the device 10.

A schematic depiction of certain elements inside the housing 86 of the oral device 10 is presented in FIG. 5. The housing 86 may include the controller 30. As discussed previously, the controller 30 may be connected to the first sensor 26 and the second sensor 82 via wires 92, 94 in tubes 88, 90. The housing 86 may also include a motor 120 electronically coupled to the rod 18. In some embodiments, the motor 120 can be configured to rotate the rod 18 to anteriorly advance the lower section 16 from the first position to the second position. The motor 120 may be any type of motor, particularly, any type of electric motor, more particularly, a stepper motor. A stepper motor may advantageously allow the lower section 16 to be advanced from one discrete position to another. This type of motor is particularly advantageous in connection with the oral device 10 as it may be configured to control the movement of the lower section 16 from the discrete first position to the discrete second position instead of allowing for continuous movement. The motor 120 may also be configured to rotate the rod 18 in the opposite direction to posteriorly retract the lower section 16 from the second position to the first position.

The housing 86 may include a battery 122 positioned within the housing 86 and configured to provide power to the controller 30. In a preferred embodiment, the battery 122 is a lithium polymer (lipo) battery. This type of battery may be advantageous due to its light weight and flexibility. However, other types of batteries or power sources may be used to power the controller 30. The housing 86 may include any other electronics used in the device 10, including, for example, amplifiers, resistors, microcontrollers, motors, power sources, or any other electronics.

With reference to FIG. 6, the second position of the lower section 16 where the lower jaw 32 of the user 22 is advanced is represented. In response to the electronic output indicative of the apneic event, the lower section 16 is configured to be advanced from the first position to the second position anterior to the first position and move the lower jaw 32 of the user 22 anteriorly. In an embodiment where the device 10 includes the first sensor 26 and the second sensor 82, in response to the electronic output from the first sensor 26 or the electronic output from the second sensor 82 indicative of the apneic event, the lower section 16 is configured to be advanced from the first position to the second position and move the lower jaw 32 of the user anteriorly.

In a preferred embodiment, the sensor 26 is positioned on the exterior surface 80 of the posterior wall 50 of the upper section 14 such that the sensor 26 is posterior of the upper row of teeth 20 of the user 22, as shown in FIG. 6. Further, the housing 86 enclosing the electronics of the device 10 including the controller 30 and motor 120 is external to the oral cavity 28 when the device 10 is in use. FIG. 6 shows the second position of the lower section 16. In this preferred embodiment, to advance to the second position, the rod 18 was rotated about the first rotation point 36, moving the lower jaw 32 of the user 22 forward. This movement re-opens the airway 34 during the apneic event and allows the user 22 to resume breathing. As is shown in FIG. 6, in the second position, air is flowing through the nose 126 and mouth 128 of the user 22 and through the user’s airway 34. This position of the lower jaw 32 allows the user 22 to breathe which may terminate the apneic event before the brain alerts the user 22 to regain consciousness. In this way, the user 22 is less likely to wake up in the nighttime during an apneic event, leading to better sleep and decreased side effects from sleep apnea generally.

The representation of FIG. 7 presents the sensor 26 of the oral device 10 generating an electronic output indicative of the apneic event where the airway 34 of the user 22 is closed such that air cannot pass through the airway 34. In this embodiment, the first position of the lower section 16 is shown. In this example, the air is not passing into the user’s airway 34, causing the user 22 to partially or fully stop breathing. The decrease in or lack of vibration caused by air in the oral cavity 28 generates a response in the sensor 26. The sensor 26 generates the electronic output that is received by the controller 30. The controller 30 uses this electronic output to determine that an apneic event is occurring.

With reference to FIG. 8, the lower section 16 of the oral device 10 in the second position where the lower jaw 32 of the user 22 is advanced such that the airway 34 of the user 22 is open and air can pass through the airway 34 is represented. In a preferred embodiment, upon determining that the apneic event is occurring, the controller 30 may effectuate the motor 120 to rotate the rod 18 to the second position, advancing the lower jaw 32 of the user 22. As shown in FIG. 8, the airway 34 is open and air can pass into the airway 34 of the user 22. The advancement of the lower jaw 32 may terminate the apneic event and stop the brain from causing the user 22 to regain consciousness during sleep. In the preferred embodiment, the air may begin to pass into the user’s airway 34 causing the user 22 to begin breathing again. The increase in vibration caused by air in the oral cavity 28 generates a response in the sensor 26. The sensor 26 generates the electronic output that is received by the controller 30. The controller 30 uses this electronic output to determine that the apneic event is terminated. The controller 30 may then effectuate the motor 120 to rotate the rod 18 back to the first position, retracting the lower jaw 32 of the user 22. This cycle of automatically adjusting the lower jaw 32 position of the user 22 may continue throughout the night, treating sleep apnea.

With reference to FIG. 9, an embodiment of a method 200 of treating sleep apnea is shown. The method 200 may include the step 202 of receiving, at a controller 30, an electronic output from a sensor 26. The sensor 26 may be inserted into an oral cavity 28 of a user 22 posterior of an upper row of teeth 20 of the user 22. In a preferred embodiment, the sensor 26 is a piezoelectric sensor, more particularly, a PVDF sensor. Advantageously, the sensor 26 is a passive sensor, meaning it responds to an external signal without needing an internal power source to generate the output. In some embodiments, the sensor 26 may respond to vibrations from airflow in the oral cavity 28 of the user 22. In other embodiments, the sensor 26 may respond to pressure changes caused by airflow as a result of breathing. Advantageously, the sensor 26 is positioned posterior of the upper row of teeth 20 of the user 22 so that interference is limited and the sensor 26 is near the airway 34 of the user 22. More particularly, the sensor 26 may be a thin film 76 exposed to the oral cavity 28 of the user 22 such that vibrations from the airflow created by the user 22 in the oral cavity 28 may directly contact the sensor 26, causing an electronic output to be more efficiently and easily generated. The information discussed previously related to the sensor 26 applies equally here as well except where contradictory.

The method 200 may include the step 204 of determining that the electronic output is indicative of an apneic event. The apneic event may generally include the breathing of the user 22 partially or completely stopping during sleep. In some embodiments, the apneic event includes vibrations from airflow of the user 22 being less than a first threshold frequency value for a first period of time. When the breathing of the user 22 is partially or completely stopped, less airflow will be present in the oral cavity 28. The voltage generated by the sensor 26 may be less than when an apneic event is not occurring. When the controller 30 receives an electronic output from the sensor 26 that indicates an apneic event, for example, when the voltage generated by the sensor 26 is low, it compares the vibrations to the first threshold frequency value. If the vibrations are below the first threshold frequency value, the controller 30 may indicate that an apneic event is occurring or is about to occur. If the vibrations are above the first threshold frequency value, the controller 30 may indicate that an apneic event is not occurring or is terminated. The controller 30 may be configured to determine that an apneic event is occurring if the vibrations are below the first threshold frequency value for the first period of time. The first period of time may be greater than 3 seconds, more preferably, greater than 5 seconds, even more preferably, greater than 7 seconds. The first period of time advantageously is long enough for the controller 30 to reliably determine that the apneic event is occurring but not too long that the brain causes the user 22 to regain consciousness to continue breathing properly.

The method 200 may include the step 206 of actuating a mandibular advancement assembly 12. The mandibular advancement assembly 12 includes an upper section 14 configured to at least partially overlap the upper row of teeth 20, a lower section 16 configured to at least partially overlap a lower row of teeth 24, and a rod 18 coupling the upper section 14 and the lower section 16. The information discussed previously regarding the upper section 14, lower section 16, and rod 18 applies equally here as well except where contradictory. Upon actuation, the lower section 16 is advanced from a first position to a second position anterior to the first position to move a lower jaw 32 of the user 22 anteriorly. This movement may be caused by a motor 120 causing the rod 18 to rotate to anteriorly advance the lower section 16. Once the lower jaw 32 is moved anteriorly to the second position, the user’s airway 34 may open and the user 22 may begin to breathe normally before the brain causes the user 22 to regain consciousness.

In some embodiments, based on the electronic output indicative of the apneic event, the lower section 16 is configured to be anteriorly advanced at a rate of 0.01-3.0 mm/min, more preferably, 0.05-2.5 mm/min, even more preferably, 0.1-2.0 mm/min, even more preferably, 0.2-1.0 mm/min, even more preferably, 0.3 mm/min. In some embodiments, based on the electronic output indicative of the apneic event, the lower section 16 is configured to be anteriorly advanced 0.001-30 mm, more preferably, 0.005-20 mm, even more preferably, 0.01-15 mm, even more preferably, 1-10 mm. The rate of advancement and the amount of advancement may be customized based on user anatomy and need.

The method 200 may include the step 208 of determining that the electronic output is indicative of termination of the apneic event. The termination of the apneic event may generally include the breathing of the user 22 returning to normal after the apneic event. In some embodiments, the termination of the apneic event includes vibrations from airflow of the user 22 being greater than a second threshold frequency value for a second period of time. When the breathing of the user 22 returns to normal, an increased amount of airflow will be present in the oral cavity 28. The voltage generated by the sensor 26 may be increased during the termination of the apneic event compared to the voltage generated during the apneic event. When the controller 30 receives an electronic output from the sensor 26 that indicates termination of the apneic event, for example when the voltage generated by the sensor 26 is increased compared to the voltage generated during the apneic event, it compares the vibrations to the second threshold frequency value. If the vibrations are below the second threshold frequency value, the controller 30 may indicate that the apneic event is still occurring and the lower section 16 may remain in the second position. If the vibrations are above the second threshold frequency value, the controller 30 may determine that the apneic event is terminated. The controller 30 may be configured to determine that the apneic event is terminated if the vibrations are above the second threshold frequency value for the second period of time. The second period of time may be greater than 1 minute, more particularly, greater than 3 minutes, more particularly, greater than 4 minutes, even more particularly, greater than 5 minutes. The second period of time may advantageously be long enough where the user 22 has fully regained normal breathing.

The method 200 may include the step 210 of actuating the mandibular advancement assembly 12. Upon actuation, the lower section 16 is retracted from the second position to the first position to move the lower jaw 32 of the user 22 posteriorly. This movement may be caused by the motor 120 causing the rod 18 to rotate in a direction opposite of the direction of rotation of the rod 18 when advancing the lower section 16. For example, the rod 18 may move counterclockwise to advance the lower section 16 and clockwise to retract the lower section 16. Once the lower jaw 32 is moved posteriorly to the first position, the user 22 may continue to breathe normally. The rate of retraction and the amount of retraction may be the same as the rate of advancement and the amount of advancement.

Advantageously, the device 10 and method 200 may only adjust the lower jaw 32 of the user 22 when the apneic event is occurring and retract the lower jaw 32 upon termination of the apneic event or shortly thereafter. Thus, the lower jaw 32 is only advanced during select times throughout the night, enhancing user comfort. Further, no jaw repositioning devices are required in the morning as are typically needed for other jaw positioning devices that hold the lower jaw 32 in an advanced position continuously throughout the night. Thus, the device 10 and method 200 disclosed herein, including the sensor system and dynamic actuation mechanism, adjust the mandibular position in real time based on the user’s breathing patterns, minimizing the side effects of current devices and methods to treat sleep apnea. Further, the device 10 and method 200 disclosed herein are more scalable, comfortable, and travel-friendly than existing options.

It is to be understood that the foregoing description is of one or more embodiments of the invention. The invention is not limited to the particular embodiment(s) disclosed herein, but rather is defined solely by the claims below. Furthermore, the statements contained in the foregoing description relate to the disclosed embodiment(s) and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation. In addition, the term “and/or” is to be construed as an inclusive OR. Therefore, for example, the phrase “A, B, and/or C” is to be interpreted as covering all the following: “A”; “B”; “C”; “A and B”; “A and C”; “B and C”; and “A, B, and C.”

1. An oral device to treat sleep apnea, comprising:
a mandibular advancement assembly having an upper section configured to at least partially overlap an upper row of teeth of a user, a lower section configured to at least partially overlap a lower row of teeth of the user, and a rod coupling the upper section and the lower section;
a sensor positioned in the upper section of the mandibular advancement assembly and configured for insertion into an oral cavity of the user posterior of the upper row of teeth, wherein the sensor is configured to generate an electronic output; and
a controller electronically coupled to the sensor and configured to receive the electronic output and determine that the electronic output is indicative of an apneic event,
wherein, in response to the electronic output indicative of the apneic event, the lower section is configured to be advanced from a first position to a second position anterior to the first position and move a lower jaw of the user anteriorly.

2. The oral device of claim 1, wherein the rod is coupled to the upper section via a first rotation point that the rod is configured to rotate about to anteriorly advance the lower section from the first position to the second position.

3. The oral device of claim 2, wherein the rod is a first rod and the mandibular advancement assembly further comprises a second rod coupling the upper section and the lower section, wherein the second rod is coupled to the upper section via a second rotation point that the second rod is configured to rotate about to anteriorly advance the lower section from the first position to the second position.

4. The oral device of claim 3, wherein the upper section comprises a first upper straight segment, a second upper straight segment, and a third upper curved segment coupling the first upper straight segment and the second upper straight segment; wherein the lower section comprises a first lower straight segment, a second lower straight segment, and a third lower curved segment coupling the first lower straight segment and the second lower straight segment; and wherein the first rod is coupled to the upper section via the first rotation point on a posterior portion of the first upper straight segment, the first rod is coupled to the lower section via a first lower attachment point on an anterior portion of the first lower straight segment, the second rod is coupled to the upper section via the second rotation point on a posterior portion of the second upper straight segment, and the second rod is coupled to the lower section via a second lower attachment point on an anterior portion of the second lower straight segment.

5. The oral device of claim 3, wherein the first rod is coupled to the lower section at a first lower attachment point that is anterior of the first rotation point and the second rod is coupled to the lower section at a second lower attachment point that is anterior of the second rotation point.

6. The oral device of claim 2, wherein the rod is configured to anteriorly advance the lower section from the first position to the second position by rotating counterclockwise about the first rotation point and posteriorly retract the lower section from the second position to the first position by rotating clockwise about the first rotation point.

7. The oral device of claim 1, wherein the sensor is a passive sensor which generates the electronic output in response to vibrational airflow-induced mechanical stress.

8. The oral device of claim 7, wherein the sensor is a polyvinylidene fluoride sensor coupled to the upper section.

9. The oral device of claim 1, wherein the sensor comprises a biocompatible coating.

10. The oral device of claim 1, further comprising a housing operably coupled to the mandibular advancement assembly, the housing being configured to be positioned external to the oral cavity of the user, wherein the housing comprises the controller and a motor electronically coupled to the rod and configured to rotate the rod to anteriorly advance the lower section from the first position to the second position, and wherein the housing is coupled to the upper section via a tube.

11. The oral device of claim 10, wherein the tube is a hollow tube comprising a wire coupling the sensor to the controller.

12. The oral device of claim 10, further comprising a battery positioned within the housing and configured to provide power to the controller.

13. The oral device of claim 1, further comprising an upper customizable liner removably received by an interior of the upper section and configured to mate with the upper row of teeth, and a lower customizable liner removably received by an interior of the lower section and configured to mate with the lower row of teeth.

14. The oral device of claim 1, wherein the sensor is positioned adjacent to a posterior wall of the upper section.

15. The oral device of claim 14, wherein the sensor is coupled to an exterior surface of the posterior wall of the upper section and is configured for exposure to the oral cavity of the user.

16. The oral device of claim 1, wherein the sensor is a first sensor and the oral device further comprises a second sensor positioned in the upper section of the mandibular advancement assembly and configured for insertion into an oral cavity of the user posterior of the upper row of teeth, wherein the second sensor is configured to generate an electronic output, the controller is electronically coupled to the second sensor and configured to receive the electronic output from the second sensor and determine that the electronic output from the second sensor is indicative of the apneic event, and, in response to the electronic output from the first sensor or the electronic output from the second sensor indicative of the apneic event, the lower section is configured to be advanced from the first position to the second position.

17. The oral device of claim 16, wherein the first sensor is coupled to an exterior surface of a first posterior wall of the upper section, the second sensor is coupled to an exterior surface of a second posterior wall of the upper section, and the first sensor and the second sensor are configured for exposure to the oral cavity of the user.

18. A method of treating sleep apnea, the method comprising:
receiving, at a controller, an electronic output from a sensor, wherein the sensor is inserted into an oral cavity of a user posterior of an upper row of teeth of the user;
determining that the electronic output is indicative of an apneic event; and
actuating a mandibular advancement assembly, wherein the mandibular advancement assembly comprises an upper section configured to at least partially overlap the upper row of teeth, a lower section configured to at least partially overlap a lower row of teeth, and a rod coupling the upper section and the lower section, and wherein, upon actuation, the lower section is advanced from a first position to a second position anterior to the first position to move a jaw of the user anteriorly.

19. The method of claim 18, wherein the apneic event comprises vibrations from airflow of the user being less than a first threshold frequency value for a first period of time.

20. The method of claim 19, wherein the first period of time is greater than five seconds.

21. The method of claim 18, further comprising:
determining that the electronic output is indicative of termination of the apneic event; and
actuating the mandibular advancement assembly, wherein, upon actuation, the lower section is retracted from the second position to the first position to move the jaw of the user posteriorly.

22. The method of claim 21, wherein termination of the apneic event comprises vibrations from airflow of the user being greater than a second threshold frequency value for a second period of time.

23. The method of claim 22, wherein the second period of time is greater than four minutes.

24. The method of claim 18, wherein, based on the electronic output indicative of the apneic event, the lower section is configured to be anteriorly advanced at a rate of 0.1-2.0 mm/min.

25. The method of claim 18, wherein, based on the electronic output indicative of the apneic event, the lower section is configured to be anteriorly advanced 0.01-15 mm.
